(19) United States
(12) Patent Application Publication
(10) Pub. No.: US 2002/0151517 A1
(43) Pub. Date: Oct. 17, 2002

(54) Nucleosome-based anti-tumor compositions

(75) Inventors: Vladimir P. Torchilin, Charlestown, MA (US); Leonid Z. Iakoubov, Newton, MA (US)

Correspondence Address: LEE CREWS, Fish & Richardson P.C., 225 Franklin Street, Boston, MA, 02110-2804, US

(73) Assignee: The General Hospital Corporation, a Massachusetts corporation

(21) Appl. No.: 10/118,802

(22) Filed: Apr. 9, 2002

Related U.S. Application Data
(63) Continuation of application No. 09/323,752, filed on Jun. 1, 1999, now abandoned.
Application No. 09/323,752 is a continuation of application No. 08/929,166, filed on Sep. 12, 1997, now abandoned.
(60) Provisional application No. 60/026,004, filed on Sep. 12, 1996.

(51) Int. Cl. A61K 48/00

(52) U.S. Cl. 514/44

(57) ABSTRACT

A method of inhibiting neoplastic cell growth in a mammal by administering to the mammal nucleosomes that elicit the production of antinuclear autoantibodies sufficient to inhibit neoplastic cell growth.

This application claims benefit from provisional application U.S. Ser. No. 60/026,004, filed Sep. 12, 1996.

### BACKGROUND OF THE INVENTION

This invention relates to the use of nucleosomes for the treatment and prevention of cancer.

In the course of pursuing cures for cancer, researchers have attempted to evoke an effective anti-tumor immune response in individuals suffering from various forms of the disease. For this approach to succeed, one must first identify tumor antigens that effectively stimulate the immune system. Specific antigens for certain tumors, such as melanomas, have been identified (Darrow et.al., J. Immunol. 142:3329-3335, 1989; Cox et al., Science 264:716-719, 1994). Furthermore, human carcinoma-associated antigens, which can be recognized by T cells, have been described (Kantor et al., J. Natl. Cancer Inst. 84 :1084-1091, 1992; Ioannides et al., J. Immunol. 151:3696-3703, 1993; Tsang et al., J. Natl. Cancer Inst. 87:982-990, 1995). However, the number of tumors that can be treated by vaccination with preparations of specific antigens is extremely limited. To date, a vaccine effective against many different types of malignant cells has not been successfully realized.

### SUMMARY OF THE INVENTION

The invention described herein is based on the discovery that antinuclear autoantibodies (ANAs) specifically bind nucleosomes that are present on the surface of tumor cells. These antibodies are so named because they recognize an antigen that is normally found in the nuclei of cells (“antinuclear”) and they can be self-produced (“autoantibodies”), for example in the elderly or in humans (or other animals) that have an autoimmune disease.

A monoclonal ANA, designated 2C5, was generated by standard techniques from the fusion of splenocytes obtained from a healthy, aged Balb/c mouse. This antibody was shown to react with the surface of a broad spectrum of tumor cells including those derived from human lymphoid tumors (e.g., MOLT-4, HEL 92.1.7, Raji, and U-937 cells) and non-lymphoid tumors (e.g., SK-BR3 cells (from an adenocarcinoma of the breast) and PC3 cells (from an adenocarcinoma of the prostate). Furthermore, 2C5 was shown to suppress the formation of a lymphoma in vivo. Therefore, the induction of such antibodies in vivo provides a means for preventing or treating neoplastic cell growth.

Accordingly, the invention features a method of treating neoplastic cell growth in a mammal, such as a human, by administering nucleosomes that elicit the production of antinuclear autoantibodies sufficient to inhibit neoplastic cell growth. The nucleosomes may be purified from eukaryotic cells or reconstituted in vitro, as described herein, using histones and mammalian or bacterial DNA. The nucleosomes can be administered in a substantially pure form in a physiologically acceptable carrier, diluent, or excipient, with or without an adjuvant. Alternatively, the nucleosomes can be liposome-encapsulated, for example, by the method described herein. Furthermore, administration may commence before or after the appearance of a tumor.

Also within the scope of the invention is a nucleosome-based composition for eliciting the production of antinuclear autoantibodies in a mammal. The composition consists of nucleosomes (which can be isolated from a eukaryotic cell or reconstituted in vitro) and a pharmaceutically acceptable carrier, diluent, or excipient. The reconstituted nucleosomes can contain either eukaryotic or bacterial DNA, and can be encapsulated in liposomes, for example, for administration as a vaccine.

The neoplastic cell growth prevented by or treated with the composition disclosed herein may be a malignant or benign growth. Malignant cell growth can give rise to lymphomas such as Burkitt's lymphoma, pre-B lymphoma, or histiocytic lymphoma, adenocarcinomas, for example of the breast, prostate, or kidney, erythroleukemia, thymomas, osteogenic sarcomas, hepatomas, melanomas, brain tumors, glial cell tumors, ovarian or uterine tumors, pancreatic tumors, or tumors within the stomach or gastrointestinal tract.

Individuals considered at risk for developing cancer may benefit particularly from the invention, primarily because prophylactic treatment can be begun before there is any evidence of a tumor. Individuals at risk include those with a genetic predisposition to one or more cancers and those who have been inadvertently exposed to nuclear radiation or a carcinogenic substance.

By “nucleosome” is meant any complex of histones and DNA including complete, naturally occurring nucleosomes, artificially prepared “reconstituted” nucleosomes, and antigenic portions of these nucleosomes. Nucleosomes are present naturally in the nuclei of eukaryotic cells and can be reconstituted, as described below, in vitro. Naturally occurring nucleosomes appear in sectioned tissue, when viewed with an electron microscope, as beadlike bodies on a string of DNA.

The term “reconstituted,” as used herein in reference to nucleosomes, refers to the process in which nucleosomes are artificially prepared by, for example, the salt step dialysis method described below.

Enhancing the anti-tumor potential of the immune system by immunizing the host with nucleosomes is advantageous in that it is expected to generate polyclonal antibodies that will recognize several determinants of tumor cell surface-bound nucleosomes. Thus, anti-nucleosomal autoantibodies should mediate the effector anti-tumor function of the host immune system more effectively than administration of an exogenous monoclonal antibody.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a line graph depicting the selective reactivity of the monoclonal ANA 2C5 with a nucleosome-containing preparation of nucleohistones in an enzyme-linked immunosorbant assay (ELISA). The samples tested are represented on the graph as follows: nucleohistones as ▪, single-stranded DNA as O, double-stranded DNA as Δ, a mixture of individual histones as □, and ribonucleoprotein as X.

FIG. 2 is a line graph depicting the reactivity of the monoclonal ANA 2C5 to reconstituted nucleosomes. The samples tested are represented on the graph as follows: nucleosomes reconstituted in vitro from a DNA-histone mixture using step salt dialysis as ▪, similarly treated DNA as Δ, similarly treated histones as □, and a nucleosome-free DNA-histone mixture as O.

FIG. 3 is a bar graph depicting the humoral response in C57BL/6 mice to injected nucleosomes. An ELISA was performed using plasma samples obtained 0, 5, and 12 days following injection. The wells were sensitized with 50 μg/well double-stranded DNA (Bar A), 10 μg/well total histone (Bar B), or 10 μg/well nucleohistone (Bar C), and the optical density was determined (as shown on the y axis).

FIG. 4 is a bar graph depicting the MHC non-restricted cytotoxicity of mouse splenocytes against S49 lymphoma cells after immunization with nucleochromatin.

### DETAILED DESCRIPTION

The data presented below demonstrate that nucleosomes are the target for tumoricidal ANAs and that immunization with nucleosomes can provide both humoral and cellular anti-tumor responses that increase the anti-tumor potential of the immune system. Thus, nucleosomes can serve as the basis of an anti-cancer vaccine.

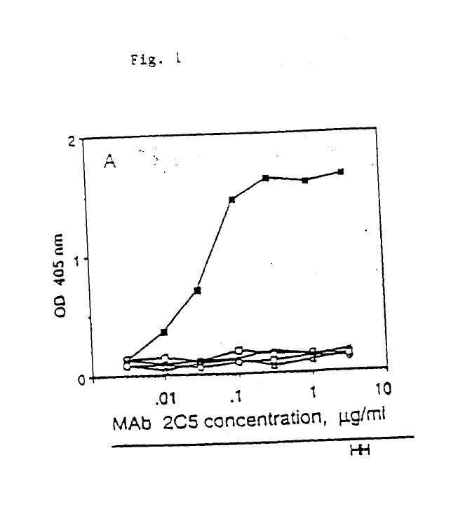

The invention is based on the discovery that an antinuclear autoantibody (ANA), 2C5, which has been shown to dramatically inhibit the development of an aggressive cancer in vivo (Torchilin et al., WO 96/00084, hereby incorporated by reference), specifically binds to nucleosomes that are present on the surface of all tumor cells examined (Torchilin et al. supra; Iakoubov et al., Immunol. Lett. 47:147-149, 1995) but not on the surface of normal, non-malignant cells. This specificity is demonstrated by Western blot analysis and by an enzyme-linked immunosorbant assay (ELISA). The reactivity of 2C5 against various potential antigenic targets is reported in Table 1 and the results of an ELISA in which a panel of different nuclear antigens was tested, is shown in FIG. 1.

Two additional ANAs, referred to as 1G3 and 4D11, were also obtained from aged, healthy Balb/c mice, and similarly have been shown to bind the surface of both human and rodent tumor cells, but not normal cells. These data are shown below in Table 2.

To conduct the initial reactivity assay, ELISA plates (Corning, New York, N.Y.) were covered with potential targets including a nucleosome-containing preparation of nucleohistone, single-stranded DNA, double-stranded DNA, a mixture of individual histones, or ribonucleoprotein (10 μg/well in phosphate buffered saline (PBS), pH 7.2) for two hours. The plates were then washed and incubated for 30 minutes with a 10% solution of heat-inactivated bovine calf serum in PBS containing 0.1% Tween 20 (PBST). This procedure effectively prevents non-specific binding. Dilutions of 2C5 or of a control isotype-matched myeloma antibody UPC10 (in the same solution; Cappel, Durham, N.C.), were added in duplicate and incubated at room temperature for 60 minutes. The bound antibody was revealed by adding peroxidase-labeled goat anti-mouse antibodies followed by substrate; visualization of absorbed goat antibodies was performed using a solution of 0.05% orthophenylenediamine hydrochloride and 0.01% hydrogen peroxide as the substrate. The reaction was stopped by adding 2.5 M sulfuric acid (50 μl/well), and the optical density was read using a microplate ELISA reader (Fisher Scientific, Pittsburgh, Pa.). In each set of experiments, a limiting value, which was taken as the mean plus 3 times the standard error of the mean (SEM) was established to permit differentiation between positive (antigen-containing) and negative serum samples. As the serum titer, the maximum dilution is taken at which the optical density of positive sample is at least 3 times higher than that of the negative sample.

The data regarding the specificity of 2C5, which was collected from the ELISA described above and from standard Western blot analysis, is shown in Table 1. The absence of reactivity with a corresponding band in the Western blot and/or reactivity within 3 standard deviations from negative control in the ELISA is indicated in Table 1 by (−). A sample was scored as positive (+++) if the signal generated was more than 10 standard deviations from the negative control in the ELISA.

TABLE 1

| Nuclear Autoantigens | | Other Potential Antiqens | |
|---|---|---|---|
| nucleohistone | +++ | myosin | − |
| ssDNA | − | β galactosidase | − |
| dsDNA | − | phosphorylase b | − |
| histones | | glutamic dehydrogenase | − |
| (individual and mixture) | − | lactate dehydrogenase | − |
| H1 peptide 144-159 | − | carbonic anhydrase | − |
| H1 peptide 204-218 | − | trypsin inhibitor | − |
| ribonucleoprotein | − | lysozyme | − |
| La/SS-B | − | aprotinin | − |
| Ro/SS-A | − | insulin | − |
| Sm | − | heparin | − |
| Jo-1 | − | dextran sulfate | − |
| scl-70 | − | heparin sulfate | − |

The monoclonal ANA 2C5 was also shown to possess nucleosome-restricted specificity when tested against reconstituted nucleosomes. Nucleosomes were reconstituted in vitro as described by Rhodes et al. (Methods Enzymol. 170:575-585, 1989). Briefly, a mixture of individual histones (50 μg/ml of each histone (H1, H2A, H2B, H3, and H4); Boehringer Mannheim, Indianapolis, Ind.) were dissolved in distilled water with 100 μg/ml purified commercial bovine thymus or bacterial DNA (Sigma Chemical Co., St. Louis, Mo.). The solution was dialyzed against 2 M NaCl for 3 hours at 4° C., followed by stepwise dialysis to 0.15 M NaCl (decrements of 0.5 M NaCl over a period of 24 hours at 4° C.). All solutions contained 1 mM EDTA and 0.1 mM phenylmethylsulfonyl fluoride.

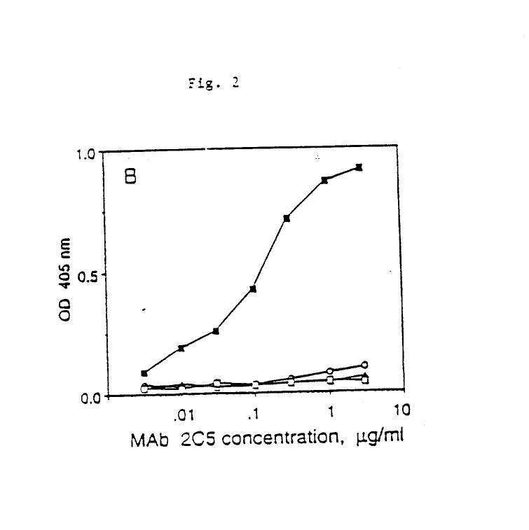

The ability of 2C5 to bind reconstituted nucleosomes was then tested. Varying concentrations of 2C5 (from approximately 0.005 to 5.0 μg/ml) were added to nucleosomes reconstituted in vitro from a DNA-histone mixture using step salt dialysis (as described above (▪)), and to similarly treated DNA (Δ), similarly treated histones (□), and a nucleosome-free DNA-histone mixture (O). A colored reaction product can be generated by tagging 2C5, for example with horseradish peroxidase, or by subsequently adding a tagged secondary antibody to the reaction. The result, as analyzed by reading the optical density (at A₄₅₀) is depicted in the line graph of FIG. 2. The ability of the 2C5 antibody to specifically bind reconstituted nucleosomes is evident by the steady increase in the optical density of the sample containing reconstituted nucleosomes with increasing concentrations of 2C5.

The ability of the ANAs 2C5, 1G3, and 4D11 to specifically bind a wide variety of human and rodent tumor cells has been demonstrated. These three ANAs were tested for their ability to bind human and rodent normal cells and human and rodent carcinomas, melanomas, sarcomas, leukemias, and lymphomas. Each of the three ANAs bound the human and rodent tumor cells, but not the normal cells. These data are shown in Table 2, where the reaction intensity is presented as a difference between flow cytometric peaks of monoclonal antibodies and a non-specific, control antibody, UPC10. The sample was scored as (+++) if the intensity was more than 3 logs from that obtained with UPC10, as (++) if the intensity was between 1.5 and 3 logs of that obtained with UPC10, (+) if the intensity was between 0.5 and 1.5 logs of that obtained with UPC10, and (−) if the intensity was less than 0.2 logs from that obtained with UPC10. Some samples were not determined (n/d).

TABLE 2

| CELLS | | 2C5 | 1G3 | 4D11 |
|---|---|---|---|---|
| Carcinomas: | | | | |
| human | | | | |
| breast ductal | BT-474 | +++ | n/d | ++ |
| colon | HT-29 | ++ | n/d | n/d |
| colon | LS-174T | ++ | ++ | n/d |
| breast | SK-BR-3 | +++ | n/d | ++ |
| adenocarcinoma | | | | |
| breast ductal | MDA-MB-134 | ++ | n/d | n/d |
| carcinoma | | | | |
| prostate carcinoma | DU145 | +++ | ++ | n/d |
| prostate | PC3 | +++ | n/d | n/d |
| adenocarcinoma | | | | |
| rodent | | | | |
| lung | LL/2 | ++ | ++ | n/d |
| squamous cell | KLN205 | ++ | n/d | n/d |
| caracinoma | | | | |
| Melanomas: | | | | |
| human | SK-MEL-5 | + | n/d | n/d |
| rodents | B16 | ++ | n/d | n/d |
| | Clone M-3 | + | n/d | n/d |
| Sarcomas: | | | | |
| human | | | | |
| osteogenic sarcoma | U-20S | +++ | +++ | n/d |
| rodent | | | | |
| osteogenic sarcoma | UMR | +++ | n/d | +++ |
| Leukemias: | | | | |
| human | | | | |
| promyeloblastic | HL60 | + | n/d | n/d |
| erythroleukemia | HEL 92.1.7 | ++ | n/d | n/d |
| rodent | L1210 | + | n/d | n/d |
| | P383 | ++ | n/d | n/d |
| | J774 | ++ | n/d | n/d |
| Lymphomas: | | | | |
| human | | | | |
| T lymphoma | MOLT4 | ++ | ++ | n/d |
| Burkitt lymphoma | Raji | + | n/d | + |
| Burkitt lymphoma | Daudi | + | n/d | n/d |
| histocytic lymphoma | U-937 | + | n/d | n/d |
| plasmocytoma | RPMI 8226 | ++ | n/d | n/d |
| rodent | | | | |
| T lymphoma | YAC-1 | +++ | n/d | n/d |
| T lymphoma | S49 | ++ | n/d | + |
| pre-B lymphoma | 7OZ/3 | ++ | n/d | n/d |
| B lymphoma | A20 | +++ | ++ | n/d |
| B lymphoma | CH1 | +++ | n/d | ++ |
| myeloma | P3X63-Ag.8.653 | + | ++ | n/d |
| plasmocytoma | MOPC 315 | ++ | n/d | n/d |
| thymoma | EL4 in culture | ++ | + | ++ |
| thymoma | EL4 from tumor | ++ | n/d | n/d |
| Normal cells: | | | | |
| human | | | | |
| PBML from fresh blood | | − | − | n/d |
| PBML in 24 hr cell | | − | n/d | − |
| culture | | | | |
| rodent | | | | |
| splenocytes, Balb/c, | | − | n/d | − |
| fresh | | | | |
| lung cells, Balb/c, | | − | − | n/d |
| fresh | | | | |
| liver cells, Balb/c, | | − | − | − |
| fresh | | | | |

To determine whether the anti-tumor potential of the immune system can be increased in non-autoimmune adult mice, nucleosomes were prepared and used to immunize these animals as follows.

Preparation of Nucleosomes

Two types of nucleosomes, one containing mammalian DNA and mammalian histones, and the other containing bacterial DNA and mammalian histones, can be reconstituted in vitro using the standard procedure of step salt dialysis described above (see also Rhodes et al., Methods Enzymol. 170:575-585, 1989). Bacterial DNA itself can exhibit an adjuvant function due to the presence of hypomethylated CpG dinucleotides, which are much less characteristic of mammalian DNA (Krieg et al., Nature 374:546-549, 1995; for review, see Krieg, J. Clin. Immunol. 15:284-292, 1995). Thus, the mammalian immune response against immunogens containing bacterial DNA may be greater than-the response to mammalian DNA.

For subsequent immunization, both preparations can be further combined with an adjuvant, such as Freund's adjuvant, or incorporated into phosphatidyl choline (PC) or PC/cholesterol liposomes as described below.

Nucleosomes can be administered directly or first entrapped within liposomes, which are artificial phospholipid nanovesicles. Liposomes can be made, for example, of pure egg lecithin, or of a mixture of lecithin and cholesterol in a 7:3 molar ratio, by e.g., the reverse phase evaporation method of Szoka et al. (Proc. Natl. Acad. Sci. USA 74:4191, 1978)). After the lipids are dried under argon and vacuum, the resulting film is dissolved in ether. For example, a film containing 16 mg of lecithin, with or without an appropriate quantity of cholesterol, is dissolved in 640 μl of ether, and supplemented with 100 to 500 μg of prepared nucleosomes (at 1 μg/μl) in phosphate buffered saline, pH 7.5. The mixture is then vortexed for 1 minute and treated in an ultrasound disintegrator (e.g., a Lab-Line Ultratip Labsonic System) at 40 W for 3-5 minutes at 4° C., and the ether is removed using a rotor evaporator.

Alternatively, nucleosomes can be entrapped within liposomes by dehydration-rehydration of vesicles according to Senior et al., Biochem. Biophys. Acta. 1003:58-62, 1989), or by prolonged co-sonication as described by Trubetskoy et al., FEBS Lett. 299:79-82, 1990). In the former procedure, 150 μl of pyrogen-free deionized water is added to the lipid film (prepared by solvent evaporation from a solution of one or more lipids in chloroform), and the film is resuspended in phosphate buffered saline, pH 7.5. Nucleosomes are incorporated by vigorous vortexing at a nucleosome:lipid weight ratio of 1:10. The final mixture is sonicated three times for one minute each at 0° C., under an argon flow, and then freeze-dried. The dry residue is reconstituted with 1 ml of pyrogen-free saline. In the latter procedure, the lipid film is resuspended in the presence of the same quantity of saline and nucleosomes by sonication for 35 to 40 minutes at 0° C., under argon flow.

The efficiency of the nucleosomal incorporation into liposomes can be determined by labeling the nucleosomes with fluorescein isothiocyanate (FITC, Sigma Chemical Co., St. Louis, Mo.) and subsequently separating the liposome-entrapped from the non-entrapped nucleosomes by Ficoll density gradient centrifugation. To accomplish this, 250 μl of a liposome-FITC-labeled nucleosome preparation is mixed vigorously with 60% Ficoll-400 in PBS (1:1 ratio, v:v), transferred to a plastic tube, and carefully layered from the top with 3 ml of a 40% Ficoll solution (in PBS) and 250 μl of PBS, without mixing the phases. The tube is then centrifuged at 35,000 rpm, for example in a Beckman ultracentrifuge, for 1 hour at −17° C. Liposomes with incorporated nucleosomes will partition into the upper layer, as will be evident from fluorescence intensity readings obtained before and after addition of a detergent, such as Triton X-100, to aliquots consisting of 10 successive fractions of 375 μl each.

The fluorescence of liposome-entrapped and non-entrapped nucleosomes can be determined, for example, using a Hitachi spectrofluorimeter, according to the manufacturer's instructions. The liposome-associated fluorescence intensity will also reflect the efficiency of nucleosome incorporation. If necessary, the composition of the liposomes can be varied to provide maximum nucleosome incorporation (see, e.g., Lesserman, Liposomes as Transporters of Oligonucleotides In “Liposomes as Tools in Basic Research and Industry,” pp. 215-223, J. R. Philippot and F. Schuber, Eds., CRC Press, 1995).

Entrapping nucleosomes within liposomes, which are then administered as described herein, offers additional advantages in that lipsomes are versatile and effective immunoadjuvants (Gregoriadis, Immunol. Today, p. 89-97, 1990; van Rooijen, Liposomes as Carrier and Immunoadjuvant of Vaccine Antigens, In “Bacterial Vaccines,” pp. 255-279, Alan R. Liss, Inc., 1990). They are considered versatile because their properties can be altered by altering their chemical and physical composition, and they have been proven effective; the immune response induced by an influenza antigen administered within liposomes was several fold greater than when administered with other adjuvants (Mbawnike et al., Vaccine 8:347-352, 1990). Furthermore, liposomes are biodegradable, non-immunogenic, less toxic and less irritating than conventional adjuvants, and they stimulate both humoral and cellular immune responses (Alving, J. Immunol. Meth. 140:1-13, 1991; Fries et al., Proc. Natl. Acad. Sci. USA 89:358-362, 1992).

Immunization

Rodents, such as C57BL/6 or Balb/c mice, can be immunized with different nucleosomal preparations, for example those combined with adjuvant or encapsulated in liposomes, according to the protocol disclosed by Mohan et al. (J. Exp. Med. 177:1367-1381, 1993). The mice are injected intraperitoneally three times, at 2 week intervals, with nucleosomes or, as a control, with PBS. When Freund's adjuvant is used, the first injection consists of nucleosomes (10 μg in 50 μl PBS/mouse) or PBS (50 μl/mouse) mixed 1:1 with complete Freund's adjuvant (Gibco Laboratories, Gaithersburg, Md.), and the two subsequent injections are administered in incomplete Freund's adjuvant. When liposome-encapsulated nucleosomes are administered, all three injections can consist of the same antigen preparation, i.e., the quantity of nucleosomes and the volume of the injection are identical to that administered with Freund's adjuvant. When administering liposome-encapsulated nucleosomes, the negative control can be liposomes that do not contain nucleosomes.

Analysis of the Humoral Immune Response

The humoral component of the immune response can be tested, for example, 7 and 12 days following the first immunization, and 5 and 9 days after the second and third immunizations. The production of nucleosome-reactive and tumor cell surface-reactive antibodies of the IgM and IgG isotypes in blood samples of individual immunized mice is examined, as is the production of these antibodies in non-immunized mice or those immunized with either adjuvant alone or liposomes alone. The pattern of nucleosome-reactive antibodies is characterized in each case using different ELISA-based systems that allow different types of nucleosome-reactive antibodies to be quantified, particularly antibodies with DNA-, histone-, and nucleosome-restricted specificities.

Blood samples from immunized mice can be screened for the presence of ANAs as follows. Approximately 5 μl of blood plasma obtained from individual, immunized mice (obtained, e.g., as described above, 7 and 12 days following the first immunization, and 5 and 9 days after the second and third immunizations) are serially diluted in 10% calf bovine sera (in PBS). The diluted samples are then tested for nuclear reactivity, as evidenced by immunofluorescent staining of commercially available Hep-2 cells (Immunoconcepts, Sacramento, Calif.). Samples from non-immunized mice can be used as negative controls, and the 2C5 antibody can be used as a positive control. The Hep-2 cells are washed 5 times with PBS, and incubated in 10% calf bovine sera (in PBS; HyClone, Logan, Utah) with either the variously diluted plasma samples or mAb 2C5 for 15 minutes. The cells are then washed twice with PBS, incubated with working dilutions of FITC-labeled F(ab)₂ fragments of goat anti-mouse IgG (whole molecule; in PBS) with 1% bovine calf sera, and washed again with PBS. The humoral immune response of immunized animals can be assessed by comparing the intensity of Hep-2 staining produced by plasma samples from these animals with the staining produced by 2C5.

Aliquots of the same diluted plasma samples (from mice immunized with various nucleosomal preparations and from non-immunized mice) that were used to stain living cells can be used to stain fixed Hep-2 cells. Before beginning this analysis, cell viability should be determined, for example by the Trypan Blue exclusion test, and should be at least 95%. The cells are washed twice with Hank's Buffered Saline Solution (HBSS), incubated for 30 minutes with plasma from immunized mice, plasma from non-immunized mice, or the monoclonal antibody 2C5 (as a positive control, at 5 μg/ml in medium containing 10% bovine calf sera), and washed twice with HBSS. The cells are then stained for 30 minutes with FITC-labeled F(ab)₂ fragments of goat anti-mouse antibody diluted 1:100 in medium containing 1% bovine calf serum. After staining, the cells are washed twice with HBSS, and fixed with 4% paraformaldehyde in PBS. All incubations are performed at 20° C. The cells may be analyzed using FACScan (Becton Dickinson, Mountain View, Calif.) and live-gated using forward and 90° scatter to exclude debris and dead cells.

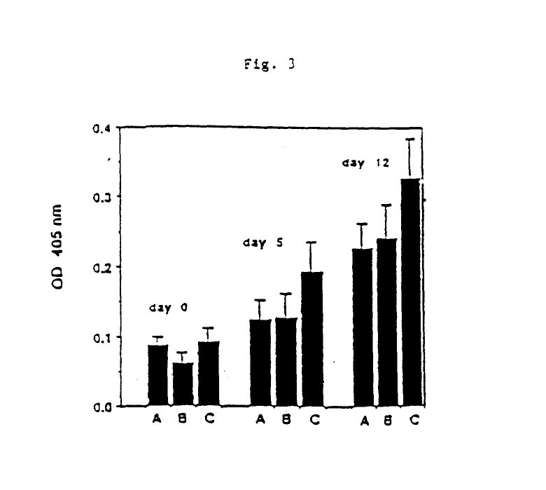

The early immune response to injection of nucleosomes was analyzed by ELISA, as follows. ELISA plates were sensitized with 50 μg/well of double-stranded DNA (Bar A in FIG. 3), 10 μg/well of total histone (Bar B in FIG. 3), or 10 μg/well of nucleohistone (Bar C in FIG. 3), washed in PBS with 0.1% Tween 20 (PBST) and incubated for 30 minutes with a 10% solution of heat-inactivated fetal calf serum in PBST to prevent non-specific binding. Plasma samples from immunized mice were diluted 1:100 in PBST and added in triplicate. After 1 hour of incubation at room temperature, the bound material was revealed by adding peroxidase-conjugated goat anti-mouse IgG for 1 hour (Cappel, Durham, NC; 1:1000 in PBST) followed by a solution of 2,2′-asino-bis(3-ethylbenz-thuazoline-6-sulfonic) acid in 0.05 M citrate buffer (pH 4.0). Hydrogen peroxide (0.01%) was used as the substrate to obtain a color reaction. The optical density of each sample was measured. As shown in FIG. 3, nucleohistones elicited the most effective immune response, with nucleosome-reactive antibodies appearing in the blood within 5 days of the initial immunization. As described herein, these antibodies specifically bind nucleosomes expressed on the surface of tumor cells but not on the surface of normal cells.

Analysis of the Cellular Immune Response

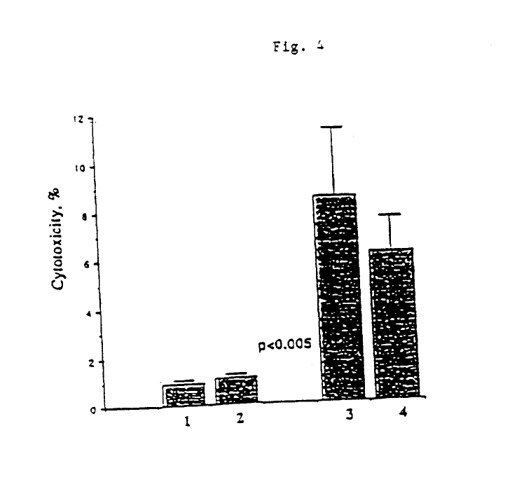

The effectiveness of the cellular immune response was also studied. The cellular component of the immune response, which is either MHC-restricted or MHC-non-restricted, can be tested by examining cellular cytotoxicity in in vitro assays in which splenocytes from immunized and control mice are used as effector cells, and 51-Cr-labeled EL4 T lymphoma cells and S49 T lymphoma cells are used as syngeneic or allogeneic targets. The tumor cells useful for studies of the cellular immune response include those from the EL4 lymphoma cell line, which originated in C57BL/6 mice treated with dimethyl benzanthracene. Inoculation with a small number of these cells leads to progressive tumor formation and subsequent death of all animals. Such aggressive tumorigenicity makes these tumor cells attractive as an experimental model. The S49 cells, which were used in the assay depicted in FIG. 4, are from a mouse lymphoma cell line that was established from a lymphoma induced in a Balb/c mouse by injection of phage and oil. These cells do not bear surface immunoglobulins.

Both EL4 T lymphoma and S49 cells are available from the American Type Culture Collection (A.T.C.C.; Rockville, Md.) under Accession Numbers TIB-39 and TIB-28, respectively.

MHC-non-restricted cytotoxicity of mouse splenocytes against S49 T lymphoma cells was demonstrated following immunization with nucleosomes, as follows. C57BL/6 mice were immunized intraperitoneally with nucleochromatin (100 μg/mouse) in complete Freund's adjuvant. Splenocytes were isolated on day 5, boosted in vitro (5% CO₂. 37° C.) with 50 μg/ml of nucleochromatin for 24 hours and, after washing, added in triplicate to the wells of a round-bottomed 96-well plate containing 51-Cr-labeled S49 T lymphoma cells (E:T=20:1). After 8 hours of incubation, the released radioactivity was quantified in a γ-counter and the degree of cytotoxicity was determined as the % lysis, according to the formula:

$$\% lysis = 100 \times \frac{observed cpm - background cpm}{total cpm - background cpm}$$

Significantly higher cytotoxicity of splenocytes from immunized mice (see column 3 of FIG. 4) versus mice injected with Freund's adjuvant alone (see column 1 and 2 of FIG. 4) was observed. The cytoxic effect could be partially inhibited when nucleosomes were present in the incubation medium throughout the experiment (columns 2 and 4 of FIG. 4).

Identification of the Cellular Subsets Responsible for Cytoxicity

To determine the mechanism and type of cellular immune response, the particular population of splenocytes must be determined. Therefore, the cytotoxicity of splenocytes from immunized mice should be tested after the depletion of different cellular subsets using complement-dependent lysis mediated by pan-T, pan-B, anti-CD4, anti-CD8, or anti-NK monoclonal antibodies (Boyle et al., J. Immunol. Meth. 15:135-146, 1977).

Analysis of the Effect of Nucleosome-based Immunization on Protection from Tumor Formation

Nucleosomal-based vaccines can be readily assessed for their effectiveness in cancer therapy. For this purpose, syngeneic tumor cells are administered to nucleosome-immunized C57BL/6 mice according to standard techniques. For example, 2×10⁴ EL4 lymphoma cells are injected intraperitoneally or 2×10⁶ B16.F10 melanoma cells are injected intravenously. The tumor-preventative effect of the immunization can be tested: (a) at the peak of the humoral IgG. antinucleosome response, (b) at the peak of the immunization-induced cellular cytotoxicity against tumor targets, and/or (c) when both components, humoral and cellular, are equally well presented. These data can be used to select an optimum protocol for immunization with nucleosomes.

B16.F10 melanoma cells are a derivative of B16 melanoma cells that have a highly metatastic potential for the lung and are available from the A.T.C.C. (Accession No. CRL-6322).

Analysis of the Effect of 2C5 Administration on the Development of a Human Tumor

To determine the effect of administration of the ANA 2C5 on human tumor cells, BT20 human breast carcinoma cells were implanted into nude mice subcutaneously and the animals were treated with four intravenous injections of 2C5 (75 μg/injection) every second day, starting on the day the tumor cells were administered. A group of control mice received similarly scheduled injections of the isotype-matched control antibody, UPC10. After 40 days, 75 percent of the treated mice were tumor-free, whereas every control mouse had developed a tumor. The average size of the tumor in the 25 percent of 2C5-treated mice that developed tumors, was only 10 to 15% as large as the tumors developed by mice that were not treated with 2C5.

Vaccination with Nucleosomes Protects Against Tumorigenesis

The effect of vaccinating mice (C57BL/6) with nucleosomes was tested using the following immunization protocol and two syngenic tumor models: EL4 T lymphoma and Lewis carcinoma. Mice were immunized with a nucleohistone preparation that contains mononucleosomes and oligonucleosomes (Sigma Chemical Co.) by intraperitoneal or subcutaneous injection, and then injected with tumor cells, as described below.

Two adjuvant protocols were used for the immunization. According to the first, nucleosomes were injected in incomplete Freunds adjuvant. According to the second, a mixture of nucleosomes and oligonucleotides containing nucleotide sequence from bacterial DNA was used (5 μg/mouse/injection). The oligonucleotides possessed strong adjuvant activity.

The mice were divided into two groups: an experimental group, in which mice were immunized with 100 μg of nucleosomes on day 0 and on day 9, and a control group that received a sham immunization consisting of PBS. Tumor cells were administered to the mice 9 days after the second immunization with nucleosomes, as follows. One group of experimental mice received an injection of EL4 T lymphoma cells (50,000 cells/mouse), and another group of experimental mice received an injection of Lewis carcinoma cells (250,000 cells/mouse). To avoid producing and observing simply a local effect, the nucleosomes and tumor cells were injected into different sites. That is, mice immunized by i.p. injection of nucleosomes received Lewis carcinoma cells by subcutaneous injection. Similarly, mice immunized by subcutaneous injection of nucleosomes received EL4 T lymphoma cells by i.p. injection.

Regardless of the route or site of administration, the development of tumors was strongly inhibited. On day 15, the average weight of the tumors that developed following administration of Lewis carcinoma cells in nucleosome-treated mice was less than one third the weight of tumors in untreated mice (i.e., PBS sham-immunized) mice. Tumors in untreated mice weighed 0.34±0.49 g, while tumors in mice treated with nucleosomes and incomplete Freunds adjuvant weighed 0.08±0.07 g, and tumors in mice treated with nucleosomes and oligonucleotides weighed 0.11±0.08 g. The development of EL4 T lymphoma was also strongly inhibited in immunized mice. In this instance, tumors in untreated mice weighed 3.3±0.49 g, but tumors in mice treated with nucleosomes and oligonucleotides weighed only 1.3±0.21 g.

Analysis of the Effect of Nucleosome-based Immunization on the Development of Established Tumors

Immunization with nucleosomes should also be effective when a tumor is already present in the host. To analyze this aspect of the invention, immunizations are performed when macroscopic tumor lesions have developed (for example, in mice on the 7th day after i.p. injection of EL4 T lymphoma cells or the 20th day after i.v. injection of B16 melanoma cells). The type of immunizing agent is chosen according to the humoral immune response and the subset of cells shown to be responsible for cytotoxicity.

Use

Skilled artisans will understand that any nuclear material that contains nucleosomes will elicit the production of antinuclear autoantibodies that specifically bind nucleosomes. This nuclear material includes, For example, nucleohistones, which are complex nucleoproteins that include the nucleosome and additional proteinaceous nuclear material, such as the DNA-binding proteins that function as transcription factors. Nuclear extract, nucleochromatin, or subnucleosomes, which are nucleosomes that have a structure that differs from that of naturally-occurring nucleosomes, can also elicit the generation of ANAs, and thus are considered within the scope of the invention.

In addition to the intraperitoneal route of administration described above, nucleosome-based vaccines can be administered intravenously, intramuscularly, transmucosally, or subcutaneously. These modes of administration can also be combined. For example, the first administration can be transmucosal and the subsequent administration can be intraperitoneal.

Vaccines can be administered in any pharmaceutically acceptable carrier or diluent, including water, normal saline, phosphate buffered saline, or a solution of bicarbonate such as 0.1 M NaHCO₃. The carrier or diluent is selected on the basis of the mode and route of administration, and standard pharmaceutical practice. Additional suitable pharmaceutical carriers and diluents, as well as pharmaceutical necessities for their use in pharmaceutical formulations, are described, for example, in Remington's Pharmaceutical Sciences, a standard reference text in the field of pharmacology.

The amount of vaccine administered will depend on the particular vaccine antigen, whether an adjuvant is co-administered, the mode and frequency of administration, and the desired effect. Each of these considerations are understood by skilled artisans. In general, the vaccine antigen of the invention (the nucleosome) is administered in amounts ranging between, for example, 1 μg and 100 mg. If adjuvants are administered with the vaccines, amounts ranging from between, for example, 1 ng and 1 mg of antigen can be used. The dosage can also be calculated empirically, for example,. based on animal studies and, expressed in terms of a patient's weight, can range from 0.2 to 200 μg/kg.

Skilled artisans will recognize that the vaccine described herein can be administered in conjunction with other methods of treatment. For example, the vaccine can be administered before, during, or after administration of chemotherapeutic agents, radiation therapy, or surgical ablation of a malignant tumor or benign growth of cells.

### OTHER EMBODIMENTS

A number of adjuvants, in addition to those described above, are known to skilled artisans and may be used to perform the immunization described herein. For example, cholera toxin (CT), the heat-labile enterotoxin of Escherichia coli (LT), or fragments or derivatives thereof having adjuvant activity, can be used for transmucosal administration. Alternatively, adjuvants such as RIBI (ImmunoChem, Hamilton, Vt.) or aluminum hydroxide can be used for parenteral administration.

Fusion proteins containing nucleosomes fused to an adjuvant (e.g., CT, LT, or a fragment or derivative thereof having adjuvant activity), are considered within the scope of the invention, and can be prepared using standard methods (see, e.g., Ausubel et al. “Current Protocols in Molecular Biology, Vol. I,” Green Publishing Associates, Inc., and John Wiley & Sons, Inc., NY, 1989). In addition, the vaccines of the invention can be covalently coupled or cross-linked to adjuvants. Methods of covalently coupling or chemically cross-linking adjuvants to antigens are described in, for example, Cryz et al. (Vaccine 13:67-71, 1994), Liang et al. (J. Immunol. 141:1495-1501, 1988), and Czerkinsky et al. (Infection and Immunity 57:1072-1077, 1989).

As stated above, the nucleosomes can be administered as a physiologically acceptable formulation containing an excipient. Examples of excipients which may be included with the formulation are buffers such as citrate buffer, phosphate buffer, acetate buffer, and bicarbonate buffer, amino acids, urea, alcohols, ascorbic acid, proteins, such as serum albumin and gelatin, EDTA, sodium chloride, polyvinylpyrollidone, mannitol, sorbitol, glycerol, propylene glycol, and polyethylene glycol (e.g., PEG-4000, PEG-6000).

It is to be understood that while the invention has been described in conjunction with the detailed description thereof, that the foregoing description is intended to illustrate and not limit the scope of the invention, which is defined by the scope of the appended claims. Other aspects, advantages, and modifications are within the scope of the following claims.

What is claimed is:

1. A method of treating an existing neoplastic cell growth in a mammal, said method comprising administering to the mammal an amount of nucleosomes effective to elicit in the mammal the production of sufficient antinuclear autoantibodies to inhibit neoplastic cell growth.

2. A method of claim 1, wherein said nucleosomes comprise mammalian DNA.

3. A method of claim 1, wherein said nucleosomes comprise bacterial DNA.

4. A method of claim 1, wherein said nucleosomes are liposome-encapsulated.

5. A method of claim 1, wherein said mammal is a human.

6. A method of claim 1, wherein said neoplastic cell growth is malignant.

7. A method of claim 1, wherein said neoplastic cell growth is benign.

8. A method of inhibiting neoplastic cell growth in a mammal at risk for neoplastic cell growth, said method comprising administering to the mammal an amount of nucleosomes effective to elicit in the mammal the production of sufficient antinuclear autoantibodies to inhibit neoplastic cell growth.

9. The method of claim 8, wherein said nucleosomes comprise mammalian DNA.

10. A method of claim 8, wherein said nucleosomes comprise bacterial DNA.

11. A method of claim 8, wherein said nucleosomes are liposome-encapsulated.

12. A method of claim 8, wherein said mammal is a human.

13. A method of claim 8, wherein said human is at risk for neoplastic cell growth.

14. A method of claim 8, wherein said neoplastic cell growth is malignant.

15. A method of claim 8, wherein said neoplastic cell growth is benign.

16. A composition for eliciting the production of antinuclear autoantibodies in a mammal, said composition comprising substantially pure nucleosomes and a pharmaceutically acceptable carrier, diluent, or excipient.

17. A composition of claim 16, wherein said nucleosomes are isolated from a eukaryotic cell.

18. The composition of claim 16, wherein said nucleosomes are reconstituted in vitro from DNA and histones.

19. The composition of claim 18, wherein said DNA is from a eukaryotic cell.

20. The composition of claim 18, wherein said DNA is from a bacterial cell.

21. A composition of claim 16, further comprising liposome-encapsulated nucleosomes.

22. A composition of claim 16, further comprising an adjuvant.
